(19) United States
(12) Patent Application Publication
(10) Pub. No.: US 2005/0207528 A1
(43) Pub. Date: Sep. 22, 2005

(54) Arrangement relating to x-ray equipment

(76) Inventor: Torbjorn Hjarn, Vaxholm (SE)

Correspondence Address: Steven S. Payne, 8027 ILIFF Drive, Dunn Loring, VA, 22027, US

(21) Appl. No.: 10/803,918

(22) Filed: Mar. 19, 2004

(51) Int. Cl. A61B 6/04; H05G 1/28

(52) U.S. Cl. 378/37

(57) ABSTRACT

To provide a good and fixed positioning and comfortable placement for a person to be examined, an arrangement is provided in a mammography apparatus. The apparatus comprises an x-ray source and an exposure area for a breast. The device includes an essentially elastic container, consisting of an x-ray permeable material, arranged in the exposure area between two compression plates. The container is arranged such that upon the addition of a medium it expands and pressurizes the breast against the compression plate

### TECHNICAL FIELD

The present invention relates to a device, and in particular a device comprising an essentially elastic container, such as a pillow made from a moldable essentially elastic material, wherein a receiving space in said elastic moldable container provides a firm and fixed positioning of an organ, such as a breast of a patient, in particular during an x-ray exposure in the course of a mammography examination.

### BACKGROUND OF THE INVENTION

During a mammography examination, the breast of the patient is arranged on a plane horizontal surface by, so-called pedals comprising one upper and one lower contact surface. Next, the x-ray permeable upper plate on the breast exerts a force. Consequently the breast remains in a fixed position by means of the pedals throughout the compression phase in order to reduce blurredness due to movement during the x-ray exposure.

During the compression phase mammography is perceived as an inconvenience to the patient as the breast compression of between 5 kg and 20 kg often results in great pain and causes discomfort to the patient.

In DE 23 35 576, for example, a liquid filled pillow is disclosed, providing a receiving space for a breast. The pillow is arranged to be filled with a breast equivalent medium, intended to compensate for the size of the breast and its density, in order to obtain the correct level of exposure and densitometer difference for the X-ray photograph from all parts of the breast, regardless of the size of the breast, when the applied force from the compression plate will push aside the breast equivalent medium along the parts of the breast having highest outward pressure. Consequently the breast equivalent medium distributes itself along the thinner parts of the breast and compensates for the parts in the breast that are thinner during the breast compression phase. Consequently, this invention relates to finding a solution to compensation by means of breast equivalent media.

Different ways of regulating the position of a breast belonging to a patient during x-ray exposure are proposed in DE 199 01 724, DE 299 08 202 and FR 2 321 263.

In U.S. Pat. No. 6,128,523 and FR 2 702 059 other devices with pillows having more or less constant volumes are proposed, which consequently are not fully adjustable. U.S. Pat. No. 6,128,523 is essentially intended for ultrasound treatment. Consequently these devices are not suitable for breasts of all sizes and shapes.

### SUMMARY OF THE INVENTION

The object of the present invention is to provide a solution to the above-mentioned problems and to provide a new type of container, which is simple to handle at the same time as it offers the patient a comfortable situation and safe fixation of the organ, such as a breast, during x-ray examination.

The above-mentioned problem is solved by means of an arrangement in a mammography equipment comprising an x-ray source and an exposure area for a breast. The device comprises an essentially elastic container composed of x-ray permeable material, arranged in the exposure area between two compression plates. The container is arranged such that upon the supply of a medium it will expand and pressurize the breast against the compression plate.

The device is arranged in an x-ray apparatus during an x-ray exposure, comprising an x-ray source and an exposure area for an organ. The elastic container is arranged in the exposure area, whereupon it expands and thus pressurizes the organ upon the supply of a medium to the container.

After the expansion of the elastic container, the device provides a contact surface between the adjustable elastic container and the organ, which exposes a space.

In one embodiment, the elastic container is a pillow arranged on a lower contact plate of a conventional mammography apparatus. A second opposite, such as one to x-rays transparent upper plate, is arranged to interact with the elastic container.

The contact plate, and consequently the receiving space of the elastic container, completely surrounds the entire organ of all sizes and shapes.

The material of the elastic container is preferably made from an adjustable, elastic, impermeable and tight fitting material of good structural strength, assuring that the above mentioned medium can be fitted in a space within the container without leakage of the same occurs. The material of the pillow is preferably made of fabric, a polymer, rubber material or the like.

Upon expansion of the elastic container, it is filled with a gaseous medium, such as air, a fluid such as water or a solid medium of smaller constituents.

The device may also include measuring equipment to survey and guide the compression force according to the pressure in the container, and a display unit for the reading of the pressure inside the container.

The elastic container may also be filled with a tempered medium, providing increased comfort.

### BRIEF DESCRIPTION OF THE DRAWINGS

Below the invention will be further described, in a non-limiting way, with reference to the appended drawings, in which:

FIGS. 1 to 3 illustrate side views of an x-ray apparatus including a first embodiment of the present invention arranged on the contact surface in different steps; and

FIG. 4 illustrates a cut along the line IV-IV in FIG. 3.

### DETAILED DESCRIPTION OF THE FORMS OF EXECUTION

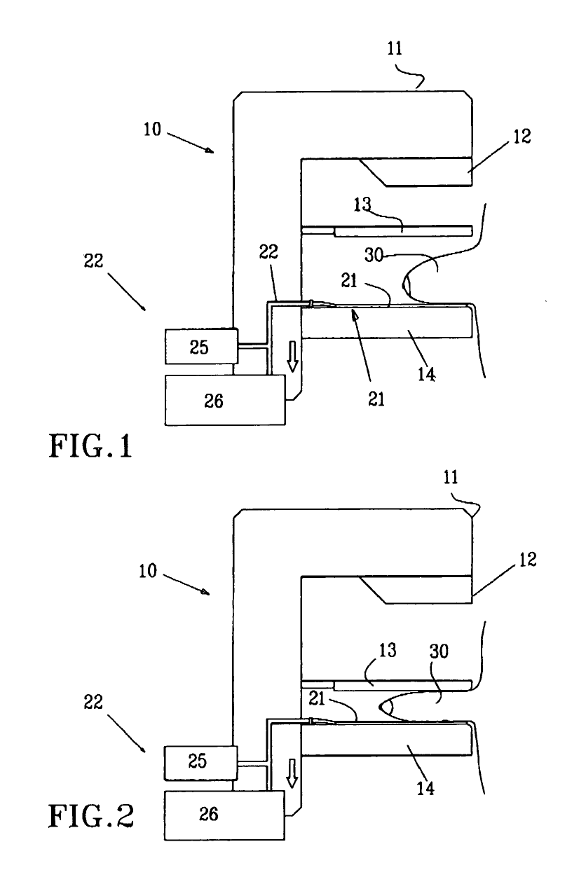

FIGS. 1 to 3 illustrate one part of an x-ray apparatus 21 including a housing 11 containing an x-ray source 12. The housing is arranged with upper and the lower horizontal plates 13 and 14, respectively, displaceable relative each other. On the lower pedal a (compression) device 20 is arranged comprising an expandable member, preferably in the form of a balloon or a pillow 21 communicating with a pump unit 22. The upper plate may encase conventional collimators (not shown) while the lower plate may encase detector units, especially in the case of a digital x-ray apparatus. The pillow is defined as an essentially elastic container, the contact surface of which is adjustable to an object, such as a breast, placed on it. An elastic container may also be defined as an expandable container having a limited expansion capability.

The pillow 21 is further arranged in such a way that it includes a tight fitting space or a chamber, which is filled with a gaseous medium, such as air. Other adequate gaseous media are also suitable for this purpose. The pillow may also be filled with a fluid or, for example a solid medium of small constituent, such as a powder.

The material of the pillow 21 is composed of a fabric, a polymer, a rubber material or the like, but is preferably made of an adjustable, elastic impermeable, tight-fitting material of good structural strength. It is important that the material of the pillow is x-ray permeable, to prevent blocking, interfering with or in other ways impairing x-rays passing through the material and preventing production of a good x-ray image.

The device according to invention further comprises connections to the pillow 21, such as valves and tubes 24, providing a tight containment of the medium, which is supplied, in addition to several inlets and outlets, intended for the medium. In one of the preferred embodiments the connections are fitted to a feeding device 25 supplying the medium. In another preferred embodiment, the device according to the invention comprises an apparatus for disposal 26 of the medium.

A conventional apparatus for mammography 10 includes a carrier providing a preferably flat horizontal surface 14, which already in present-day situations is used as a resting and contact surface for the breast 30 to be examined. The x-ray permeable upper plate 13 is arranged to exert a pressure on the breast during the breast compression phase throughout a mammography examination, as shown in FIG. 2.

Initially, the pillow 21 is essentially empty. Subsequently, the breast 30 is arranged on the pillow 21. According to a conventional mammography examination a force is exerted on the breast 30 by the upper plate 13 during a first compression phase by bringing together the plates.

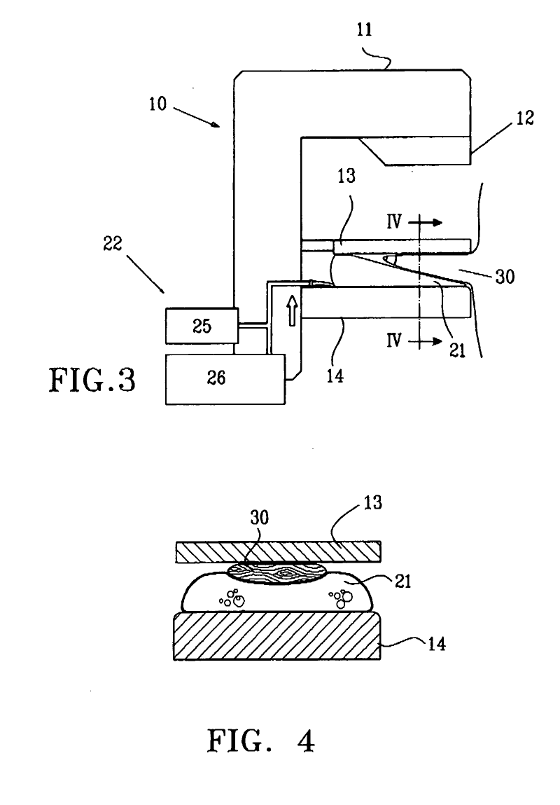

During the second phase, the pillow 21 is over pressurized by means of the pump unit 22, during which the breast maintains a firm and fixed position during the x-ray exposure according to the invention, FIG. 3, without giving the patient any discomfort due the pressure from the plates. During an overpressure the contact surface between the pillow 21 and the breast 30 will provide a receiving space, which presses tightly and firmly against the breast. According to the invention a balanced compression force uniformly distributed throughout the surface of the breast will be obtained, whereby a firm and fixed position for the breast is obtained during an x-ray exposure, as illustrated in FIG. 4.

The two breast compression phases, during which the device according to the invention is used, may be arranged in other ways than described above.

Another preferred embodiment of the device includes the connections of the pillow 21 to different gaseous mediums having the same purpose. In such a case, it is feasible that the device also includes connections for the disposal of the medium. Of course, such a disposal device is also feasible in an embodiment according to the invention, where the medium is a fluid or a solid medium distributed as small components

In one especially preferred embodiment, the invention may comprise a measuring apparatus (not shown) and/or a display unit, to measure or control the compression force by reading the pressure inside the pillow 21. A trigger unit may also be attached to the device according to the present invention, for the guidance of the breast compression.

In one preferred embodiment, it is possible to temper the medium, in order to provide a more comfortable receiving space for the breast.

It may also be possible to place the pillow in the upper plate or both in the upper and lower plates. In either case, the pillow and the medium it is filled with must be permeable to x-rays.

The present invention may, as described above, be combined with a conventional mammography apparatus intended for similar uses. The invention may also be used under different circumstances not related to an existing mammography apparatus, and thus may be incorporated in a novel embodiment of mammography equipment and apparatus used as such.

The invention is not limited to the embodiments mentioned above but may be varied in a number or different ways, for example, by the combination of two or more of the disclosed embodiments, without departing from the scope of the invention as laid out in the following claims, the device and the method of approach may be implemented in a number of different ways depending on the field of application, functional units and demands etc.

1. An arrangement in a mammography equipment, providing a well defined and comfortable positioning and fixation of a breast, the equipment including an x-ray source and an area of exposure for a breast, wherein the device includes an essentially elastic container, consisting of an x-ray permeable material, arranged in said exposure area between two compression plates, said container being arranged such that upon the supply of a medium it expands and pressurizes said breast against said compression plate.

2. An arrangement according to claim 1, wherein a contact surface between the container and the breast exhibits a receiving space such that the compression force is essentially uniformly distributed all over the breast.

3. An arrangement according to claim 1, wherein the container is a pillow, arranged on a lower plate in the exposure area.

4. An arrangement according to claim 1, wherein one contact surface of the container completely or partly surrounds the breast.

5. An arrangement according to claim 4, wherein container is made from a fabric, a polymer or/and rubber material.

6. An arrangement according to claim 1, wherein the medium is gaseous.

7. An arrangement according to claim 6, wherein the medium is air.

8. An arrangement according to claim 7, wherein the medium is a fluid or a solid medium of smaller components.

9. An arrangement according to claim 8, wherein the medium is water.

10. An arrangement according to claim 1, wherein the device includes attachments to the container, for a tight containment of said supplied medium and one or more inlets and outlets intended for the medium.

11. An arrangement according to claim 10, wherein the inlet of the container is attached to a controllable compressor.

12. An arrangement according to claim 11, wherein the device includes a connection for a device for the disposal of the medium.

13. An arrangement according to claim 11, wherein the device includes a measuring apparatus to measure and guide the compression force by means of the pressure in the container.

14. An arrangement according to claim 1, wherein the device includes a display unit for reading the pressure inside the container.

15. An arrangement according to claim 1, wherein the container is filled with a tempered medium.

16. A compression procedure during an x-ray exposure in an x-ray apparatus, for a well defined and comfortable positioning and fixation of a breast, whereby said breast is arranged on an essentially elastic container, consisting of an x-ray permeable material, arranged in an exposure area, wherein the container expands when supplied by a medium, whereby the organ is fixed, positioned and pressurized.
